(19) United States
(12) Patent Application Publication
(10) Pub. No.: US 2006/0036130 A1
(43) Pub. Date: Feb. 16, 2006

(54) Endoscopic working channel and method of making same

(75) Inventor: Craig S. Fitzpatrick, Phoenix, AZ (US)

Correspondence Address: LaValle D. Ptak, 28435 N. 42nd St., Ste. B, Cave Creek, AZ, 85331, US

(73) Assignee: International Polymer Engineering, Inc.

(21) Appl. No.: 10/918,594

(22) Filed: Aug. 12, 2004

(51) Int. Cl. A61B 1/00

(52) U.S. Cl. 600/153; 600/920

(57) ABSTRACT

An endoscopic working channel is made of an inner length of sintered polytetraflouroethylene heat bonded to an outer tubular length of sintered expanded polytetraflouroethylene.

### RELATED PATENT

This patent application is related to the subject matter of U.S. Pat. No. 5,885,209, assigned to the same assignee as this application.

### BACKGROUND

The device of the present invention relates generally to the field of endoscopy, which includes the use of tubular structures inserted intraluminally into a mammalian body cavity for visualizing, biopsing, and treating tissue regions within the mammalian body. Most endoscopes currently include at least one of a plurality of working channels which extend along the length of the endoscope to provide access to body tissue within the mammalian body cavity. These working channels typically include a rigid non-bendable section and a flexible bendable section. These channels allow for air insufflation, water flow, suction, and biopsies. Conventional endoscopes utilize a wide variety of materials for the working channels, but all conventional endoscopes require the endoscopic working channel to be an integral part of the endoscope.

Because endoscopes are subjected to repeated use and are required to follow tortuous pathways within the body, a frequent cause of failure of the endoscope working channel is the bending, kinking or fracture of a section of the working channel. This renders the endoscope useless until it is repaired. Unfortunately, repair of the endoscopic working channel requires disassembly of the endoscope and replacement of the endoscope working channel.

The endoscopic working channel of U.S. Pat. No. 5,885,209 is designed to be retrofitted as a replacement bendable section of the working channel of an endoscope. The structure of the endoscopic working channel of U.S. Pat. No. 5,885,209, however, is relatively complex and is relatively expensive to manufacture.

### BRIEF DESCRIPTION OF THE DRAWINGS

FIG. 1 is a perspective view of an embodiment of the invention illustrating steps in its manufacture;

FIG. 2 is a perspective view of another stage of the manufacture of the device of FIG. 1;

FIG. 3A is a side view of a stage of the manufacture of the device of the embodiment shown in FIGS. 1 and 2;

FIG. 3B is a side view of a portion of a completed device of an embodiment of the invention; and

FIG. 4 is a process flow diagram illustrating a method used to manufacture the device of FIGS. 1, 2 and 3.

### DETAILED DESCRIPTION

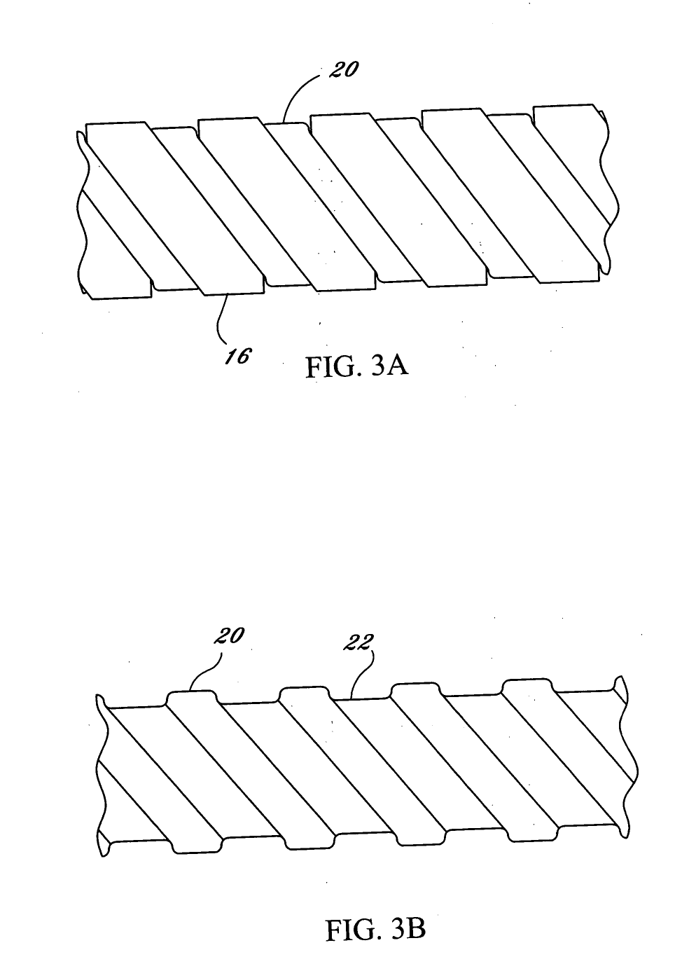

Reference now should be made to the drawings, in which the same reference numbers are used throughout the different figures to designate the same or similar components. FIGS. 2 and 3B are directed to an embodiment of an endoscopic working channel which includes an inner tubular structure 12 fabricated from sintered, non-expanded polytetraflouroethylene (PTFE). Typically, the density of this inner tubular structure is in the range of 1.8 to 2.2 g/cc. Directly over this inner (interior) PTFE tube 12 is an outer (external) tube 14 made of expanded polytetraflouroethylene (ePTFE) having a convoluted spirally or helically configured outer surface with raised portions 20 and recessed portions 22, as shown most clearly in FIGS. 2 and 3B. The finished product has the opposing ends of the tubes 12 and 14 cut co-planar to a plane which is perpendicular to the central axis of the tubes 12 and 14.

One or both of the opposing ends of the tubes 12 and 14 may be chemically etched using an etchant suitable for use with polytetraflouroethylene (PTFE), such as that sold under the trademark “FLUROETCH” or “TETRAETCH” (W.L. Gore Associates). Chemical etching facilitates subsequent adhesive bonding of the etched end with the tip of the endoscope. The end of the working channel which is intended to be the distal end of the channel may be chemically etched in order to increase the capacity of the tubes 12 and 14 to accept an adhesive bond with a distal section of an endoscope. The second or opposite end of the tubes 12 and 14, specifically that end which is intended to be the proximal end of the endoscope working channel, is not etched, as it typically is mechanically coupled to a proximal section of an endoscope. When the endoscope working channel depicted in FIGS. 2 and 3B is intended to be positioned in an intermediate region of the working channel, both the first and second ends of the endoscope working channel shown in FIGS. 2 and 3B preferably are chemically etched in order to increase their capacity to be adhesively bonded to the pre-existing working channel of the endoscope.

The convolutions which are formed by the raised and recessed portions 20 and 22, respectively, of the outer tube 14 made of expanded polytetraflouroethylene (ePTFE) permit the endoscope working channel to be twisted and bent, and still conform to its interior circular cross section provided by the PTFE inner tube 12. By making the outer tube 14 of ePTFE, the poracity of the ePTFE material provides flexibility of the overall assembly without kinking, and functions like a steel spring. In addition, the ePTFE material of the tube 14 also functions as a good insulator for the completed structure. The outer ePTFE layer 14, bonded to the inner tube 12 of PTFE, prevents the inner tube 12 from kinking when the composite assembly is bent and twisted in its use as an endoscope working channel.

As mentioned previously, both of the tubes 12 and 14 in finished assembly are sintered, that is they have been heated to a temperature above the melting point (typically around 320° C.) and then allowing the material to cool. The sintering of the two tubes 12 and 14, however, takes place at two different times during the fabrication of the assembly shown in FIGS. 2 and 3B.

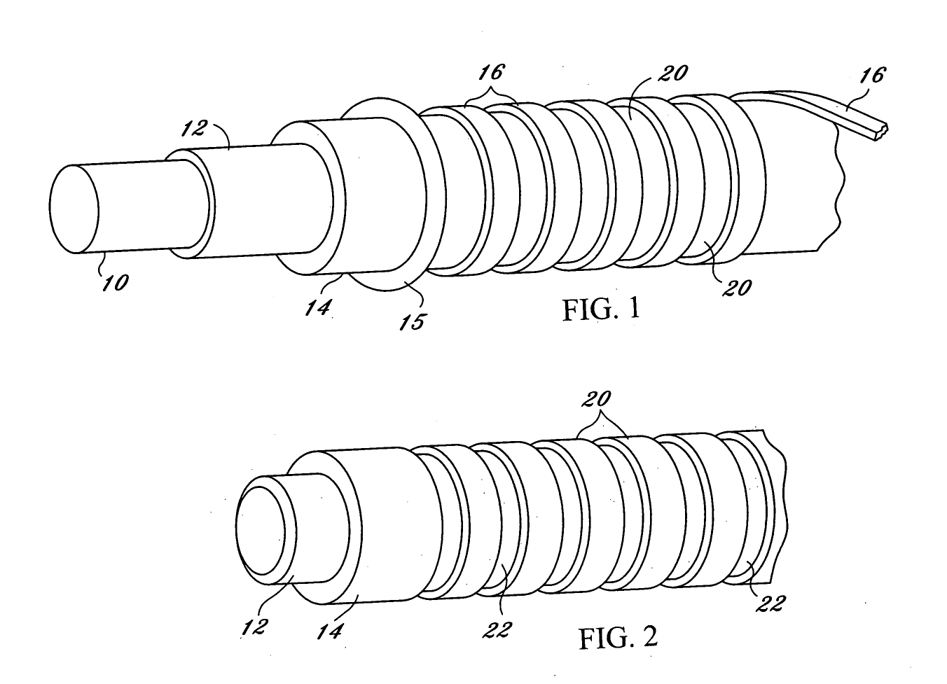

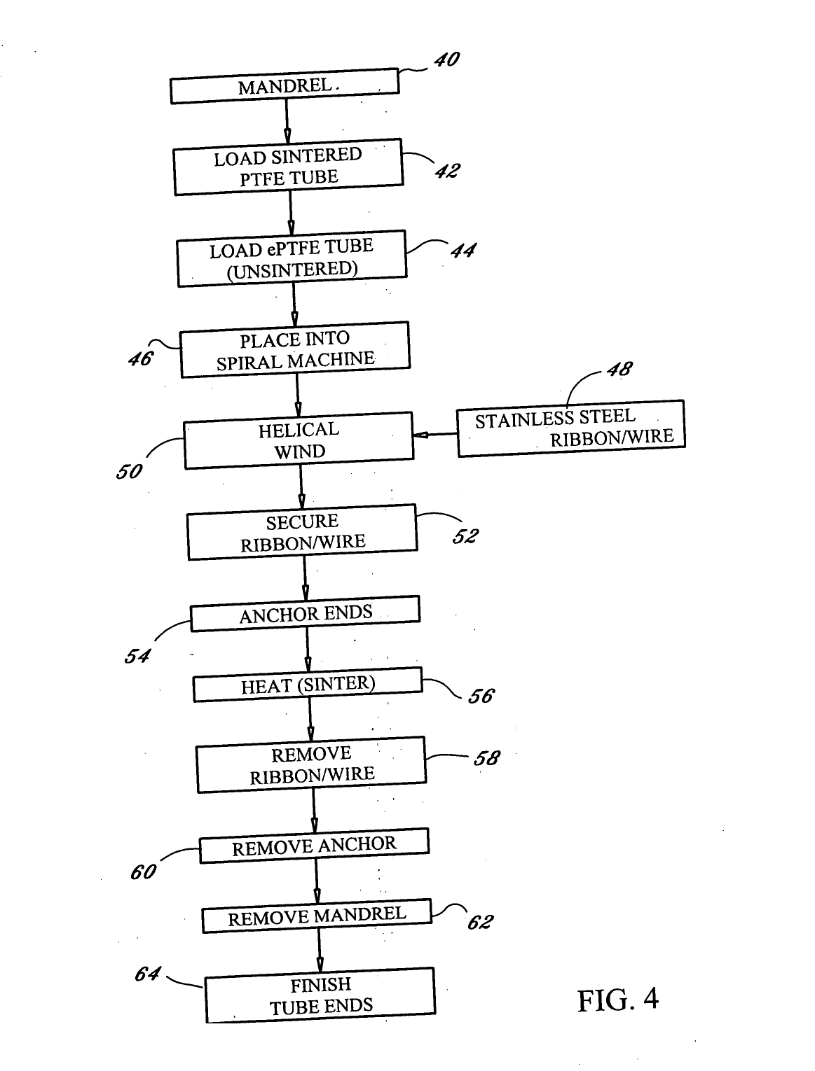

Reference now should be made to FIGS. 4 and 1 in explaining the manner in which the endoscopic working channel is manufactured. The first step (40 in FIG. 4) is to provide a mandrel 10, which is either a rod or tube made of stainless steel, brass or aluminum having a length which, preferably, is greater than the length of the finished endoscopic working channel to be made by the process. The rod 10 is a straight rod, the ends of which extend beyond the length of the endoscopic working channel to be manufactured. The rod is designed to be mounted for rotation in a spiral winding machine.

Before placing the assembly in a winding machine, however, the sintered PTFE tube 12 is loaded onto the rod 10 (step 42 of FIG. 4) by simply sliding it onto the rod 10 from one end toward the other. Once the sintered inner PTFE tube 12 is in place on the rod 10, the outer un-sintered ePTFE tube is loaded over the tube 12 at step 44, by sliding it over the tube 12. It should be noted that the inner diameter of the tube 14 is equal to or slightly greater than the outer diameter of the tube 12, and that the inner diameter of the tube 12 is equal to or slightly greater than the outer diameter of the mandrel rod 10.

After the tubes 12 and 14 have been loaded on the mandrel 10, as generally illustrated in FIG. 1, the assembly is placed into a spiral machine as shown at step 46 in FIG. 4. At this point, it should be noted that the tubing 14 not only is porous in nature (ePTFE), but that it also is un-sintered.

After the assembly is placed into the spiral machine at step 46, helical or spiral windings at step 50 (FIG. 4) of either stainless steel, brass, or aluminum wire or ribbon wire, shown at 48 in FIG. 4, is tightly wound in a spiral or helical pattern on the mandrel and then over one end of the assembly (the left-hand end as shown in FIG. 1) toward the right-hand end onto the other end of the mandrel 10. The wire 16 may have either a circular cross-section or a rectangular cross-section, as shown. The winding of the wire or ribbon wire 16 is done with sufficient pressure so as to compress the portions of the ePTFE tube 14 beneath the wire as the winding takes place with non-compressed spaces between adjacent turns of the wire 16. This, in turn, applies pressure between the inner diameter of the tube 14 and the outer diameter of the sintered tube 12, and also produces some compression of the pores of the outer ePTFE tube 14 beneath each of the turns of the wire wrap.

The wire wrap 16 is shown in both FIGS. 1 and 3A, and serves both to maintain the dimensional aspect of the tubing 14 and provides some radial compression to assist in the bonding of the tubes together. In addition, the wire 16 is made of heat-conductive material (particularly when stainless steel is used); so that the wire 16 facilitates the subsequent heating steps. Other wire shapes in addition to those described above could also be used.

After the helical or spiral winding at step 50, the wire 16 is secured at the ends on the mandrel 10 at step 52 by means of a removable tape, or any suitable material (not shown), to hold it in place during the final sintering steps of the method of fabrication of the endoscope working channel. In addition to the spiral wire or helical wire 16, brass wire or rings 15 are secured around the exterior of the tube 14 adjacent both ends, to further secure the wire wrap 16 to the tubing 14 and to prevent longitudinal retraction of the outer tubing 14 during the next processing step.

As mentioned above, the melting point of polytetraflouroethylene (PTFE and expanded polytetraflouroethylene (ePTFE) is approximately 320° C. Once the ends of the wire 16 and of the tubing 14 are secured by the brass wire 15 at step 54 (FIG. 4), the entire assembly, including the mandrel 10, is removed from the winding machine and placed in a heat sintering oven (step 56 of FIG. 4), which may be in the form of a convection air oven or a furnace at a processing temperature sufficiently high to exceed the 320° C. melting point of the expanded polytetraflouroethylene (ePTFE) material. Induction heating ovens also may be used, provided the temperatures to which the expanded polytetraflouroethylene (ePTFE) is subjected exceed the 320° sintering temperature of this material. The time duration for the sintering process of the tube 14 is approximately 1 to 2 minutes duration per foot of the assembly. This time, however, may be varied in accordance with the particular parameters of the oven used and the manner in which heat is applied to the assembly during the sintering process.

After the sintering process has been completed at step 56, and the assembly shown in FIG. 1 has been allowed to cool, the brass wire rings 15 are removed from both ends of the assembly. The wire 16 then is unraveled and discarded (step 58 of FIG. 4). The completed assembly has the configuration shown in FIGS. 2 and 3B. At this time, the mandrel 10 is removed at step 62; and the two ends of the assembly are finished at step 64 in the manner described previously.

Although the embodiment which has been described above includes the winding of a wire and its subsequent removal to form a convoluted exterior configuration of the completed assembly, some applications require an exterior surface which is relatively smooth, that is non-convoluted. In such a case, no wire would be wound about the exterior of the expanded polytetraflouroethlene tube 14 prior to the final sintering process. If such convolutions are not desired, steps 46,50,52, and 58 of the process described above and illustrated in FIG. 4 would be eliminated. The remaining steps, however, still apply to form a completed assembly having the configuration of FIG. 2, but without any of the convolutions 26/22 shown therein. The outer tube 14 would have a uniform outer diameter in such a case.

To produce an assembly having a smooth outer diameter, the first step (40 in FIG. 4) is to provide a mandrel 10, as in the case of the embodiment described above. Again, the mandrel may be a rod or tube made of stainless steel, brass or aluminum having a length which preferably is greater than the length of the finished endoscope working channel to be made by the process. A sintered PTFE tube 12 then is placed on the mandrel at 42, followed by loading of an outer un-sintered ePTFE tube over the tube 12 at Step 44, as indicated in FIG. 4. Steps 48,50,52 then are eliminated. The anchoring of the ends, as shown at Step 54 in FIG. 4, is provided, however, to prevent longitudinal shrinkage of the outer ePTFE tube during the next sintering step, which is shown as the heat (sinter) at 56. Again, for a smooth or non-convoluted surface, there is no wire so Step 58 is eliminated, or is not present. After the sintering step at 56, the anchor is removed at 60, the mandrel is removed at 62, and the tube ends are finished at 64, as described in conjunction with the helical wire wound embodiment above.

It should be noted that the range of the outer tubing diameter of the tube 14 which is developed with the process described above is between 1 mm and 8 mm in diameter. The typical inner diameter of the tube 12 (also the outer diameter of the mandrel 10) is from 0.8 mm to 6 mm.

The foregoing description of an embodiment of the invention is to be considered as illustrative and not as limiting. Various changes and modifications will occur to those skilled in the art for performing substantially the same function, in substantially the same way, to achieve substantially the same result without de parting from the true scope of the invention as defined in the appended claims.

What is claimed is:

1. A method of making an endoscopic working channel including: placing a first fixed length of tubing made of sintered non-expanded polytetraflouroethylene (PTFE) on a mandrel; placing a second fixed length of tubing made of un-sintered expanded polytetraflouroethylene (ePTFE) over the first fixed length of tubing; spiral winding a wire about the exterior of the second fixed length of tubing to form an intermediate assembly; heating the intermediate assembly to sinter the second fixed length of tubing and bond the first and second fixed of lengths of tubing together; and removing the wire from the exterior of the second fixed length of tubing.

2. The method according to claim 1 wherein placing the first fixed length of tubing on the mandrel comprises sliding the first fixed length of tubing over a mandrel having a length greater than the length of the first fixed length of tubing.

3. A method according to claim 2 wherein the mandrel is made of heat conductive material.

4. A method according to claim 3 further including removing the mandrel from the interior of the first fixed length of tubing after the wire is removed from the exterior of the second fixed length of tubing.

5. The method according to claim 4 wherein spiral winding a wire about the exterior of the second fixed length of tubing comprises spiral winding the wire with a predetermined spacing between each turn of the spiral.

6. The method according to claim 5 wherein the winding of wire about the exterior of the second fixed length of tubing comprises winding the wire with a sufficient pressure to compress the portions of the second fixed length of tubing located beneath the wire.

7. The method according to claim 6 further including the step of finishing the ends of the bonded first and second fixed lengths of tubing after removal of the mandrel.

8. A method according to claim 1 further including removing the mandrel from the interior of the first fixed length of tubing after the wire is removed from the exterior of the second fixed length of tubing.

9. The method according to claim 8 further including the step of finishing the ends of the bonded first and second fixed lengths of tubing after removal of the mandrel.

10. The method according to claim 1 wherein spiral winding a wire about the exterior of the second fixed length of tubing comprises spiral winding the wire with a predetermined spacing between each turn of the spiral.

11. The method according to claim 10 wherein the winding of wire about the exterior of the second fixed length of tubing comprises winding the wire with a sufficient pressure to compress the portions of the second fixed length of tubing located beneath the wire.

12. A method according to claim 1 wherein the mandrel is made of heat conductive material.

13. The method according to claim 12 wherein the winding of wire about the exterior of the second fixed length of tubing comprises winding the wire with a sufficient pressure to compress the portions of the second fixed length of tubing located beneath the wire.

14. The method according to claim 13 wherein spiral winding a wire about the exterior of the second fixed length of tubing comprises spiral winding the wire with a predetermined spacing between each turn of the spiral.

15. An endoscopic working channel capable of retrofit into a pre-existing endoscope including in combination: an inner sintered polytetraflouroethylene tubular member having an, internal diameter and an external diameter and having first and second opposing ends; and an outer tubular member over the interior tubular member and made of sintered expanded polytetraflouroethylene with first and second opposing ends and having an internal diameter substantially the same as the external diameter of the inner polytetraflouroethylene tubular member, with the outer expanded polytetraflouroethylene tubular member bonded to the inner polytetraflouroethylene tubular member.

16. The endoscopic working channel according to claim 15 wherein the outer tubular member is heat bonded to the inner tubular member.

17. An endoscopic working channel according to claim 16 wherein the external expanded polytetraflouroethylene tubular member is sintered onto a previously sintered polytetraflouroethylene inner tubular member.

18. An endoscopic working channel according to claim 15 the outer expanded polytetraflouroethylene tubular member has a convoluted outer surface.

19. An endoscopic working channel according to claim 18 wherein the convolutions in the outer surface of the outer expanded polytetraflouroethylene tubular member are in a continuous spiral or helical pattern.

20. The endoscopic working channel according to claim 17 wherein the outer tubular member is heat bonded to the inner tubular member.

21. An endoscopic working channel according to claim 18 wherein the external expanded polytetraflouroethylene tubular member is sintered onto a previously sintered polytetraflouroethylene inner tubular member.

22. An endoscopic working channel according to claim 15 wherein the external expanded polytetraflouroethylene tubular member is sintered onto a previously sintered polytetraflouroethylene inner tubular member.

23. A method of making an endoscopic working channel including: placing a first fixed length of tubing made of sintered non-expanded polytetraflouroethylene (PTFE) on a mandrel; placing a second fixed length of tubing made of un-sintered expanded polytetraflouroethylene (ePTFE) over the first fixed length of tubing to form an intermediate assembly; and heating the intermediate assembly to sinter the second fixed length of tubing and bond the first and second fixed of lengths of tubing together.

24. The method according to claim 23 wherein placing the first fixed length of tubing on the mandrel comprises sliding the first fixed length of tubing over a mandrel having a length greater than the length of the first fixed length of tubing.

25. A method according to claim 24 wherein the mandrel is made of heat conductive material.

26. A method according to claim 25 further including removing the mandrel from the interior of the first fixed length of tubing after the first and second fixed lengths of tubing are bonded together.

27. The method according to claim 26 further including the step of finishing the ends of the bonded first and second fixed lengths of tubing after removal of the mandrel.

28. A method according to claim 23 wherein the mandrel is made of heat conductive material.

29. A method according to claim 23 further including removing the mandrel from the interior of the first fixed length of tubing after the first and second fixed lengths of tubing are bonded together.
